(19) United States
(12) Patent Application Publication
(10) Pub. No.: US 2026/0242692 A1
(43) Pub. Date: Aug. 20, 2026

(54) UNSATURATED ETHER FRAGRANCE PRECURSORS

(71) Applicant: Givaudan SA, VERNIER (CH)

(72) Inventors: Nicolas COCITO ARMANINO, Kemptthal (CH); Felix FLACHSMANN, Kemptthal (CH); Marc LINIGER, Kemptthal (CH)

(21) Appl. No.: 19/471,181

(22) PCT Filed: Mar. 26, 2024
(86) PCT No.: PCT/EP2024/058051

§ 371 Date: Sep. 30, 2025

(30) Foreign Application Priority Data
Mar. 30, 2023 (GB) 2304737.6
Jul. 11, 2023 (GB) 2310625.5

(51) Int. Cl. C11B 9/00; A61K 8/36; A61K 8/46; A61Q 5/02; C11D 3/50

(52) U.S. Cl. CPC C11B 9/0011; A61K 8/36; A61K 8/466; A61Q 5/02; C11B 9/0019; C11D 3/50

(57) ABSTRACT

There is provided the use of a compound according to formula (I)

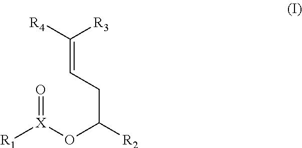

as fragrance precursor which is able to release a fragrance having aldehydic fragrance notes. There is further provided a fragrance composition and a consumer product comprising said compound, as well as the compound as such.

### TECHNICAL FIELD

The present invention relates generally to fragrance precursors, which are able to release fragrant compounds. The invention also relates to perfume preparations and consumer products containing said precursors. The invention further relates to methods of making said perfume precursors, perfume preparations and consumer products, as well as the use of said perfume precursors and perfume preparations in consumer products, such as personal care and household care products.

### BACKGROUND

Perfumed consumer products such as cleaning or laundry products comprising fragrances are well-known in the art. However, it is known that fragrances can be altered through degradation caused by interaction with air or when incorporated in certain consumer product bases, where alkalinity, acidity, the presence of oxidizing agents, such as hypochlorite salts, or other base components may lead to chemical degradation of the fragrance. In addition, volatile fragrances tend to be dissipated with time. Furthermore, when used in cleaning or laundry products, the deposition of the fragrance on a treated substrate is diminished by the washing and/or rinsing procedure.

Nevertheless, it is desired by consumers to have products that can be stored over time and still giving a constant perfume impression. In particular, the impact of volatile components is to be retained. Furthermore, it is desired that such products create a long-lasting pleasing fragrance slowly emitting from the treated substrate over time.

To address these needs, fragrance precursors can be used, which are substances that are essentially odorless themselves, but which, in particular circumstances, will decompose to release the fragrant molecule.

There are several classes of known precursors which release fragrant molecules upon activation, such as hydrolysis, temperature change, oxygen, action of light and enzymes. For example, WO2012085287 reports a group of precursors able to release a fragrance by spontaneous air oxidation. In WO2007143873 another group of precursors is described which can be cleaved by hydrolysis.

In general, precursors do show different stability, and they release the fragrant molecule under different conditions. For example, there is still need to provide further precursor systems to improve fragrance effects on dry fabric after some days. It is therefore desirable to provide a new or improved system which is capable of releasing fragrance over an extended time period and providing a long-lasting release of the fragrance.

### SUMMARY

In accordance with a first aspect of the present invention there is provided a use of a compound as fragrance precursor which is capable of releasing fragrance over an extended time period and providing a long-lasting release of said fragrance.

In accordance with a second aspect of the present invention there are provided compounds as precursors.

In accordance with a third aspect of the present invention there is provided a method to release a fragrance.

In accordance with a fourth aspect of the present invention there are provided methods of making said perfume precursors, perfume preparations and consumer products.

In accordance with a fifth aspect of the present invention there are provided perfume preparations and consumer products comprising said compounds.

In accordance with a sixth aspect of the present invention there is provided the use of said perfume precursors and perfume preparations in consumer products, such as fabric care, personal care and household care products.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that simple homoallyl esters, carbonates, carbamates and sulfonates can serve as fragrance precursors able to release fragrance. These fragrance precursors provide a delayed release of the fragrance over a longer period of time than by the use of fragrance compounds as such.

There is therefore provided herein a use of a compound according to formula (I)

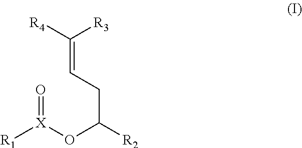

as fragrance precursor
wherein
X is selected from C and S(═O);
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me.

For example, there is provided the use of the compound of formula (I) as fragrance precursor wherein the compound is the compound according to formula (Ia)

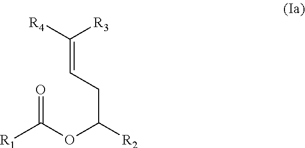

wherein
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me.

The compound of formula (Ia) corresponds to the compound of formula (I), wherein λ is C.

The compound of formula (Ia) encompass homoallyl esters, carbonates and carbamates.

For example, there is provided the use of the compound of formula (I) as fragrance precursor wherein the compound is the compound according to formula (Ib)

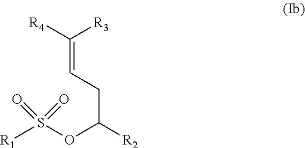

wherein
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me.

The compound of formula (Ib) corresponds to the compound of formula (I), wherein X is S(═O). The compound of formula (Ib) encompass sulfonates.

For example, the compound of formula (Ib) is a mesylate, wherein R₁ is methyl, or a tosylate, wherein R₁ is tolyl, and wherein R₂ to R₄ is as defined above.

The compounds of formula (Ia) and (Ib) are subgroups of the compound of formula (I). Accordingly, the description related to the compound of formula (I) applies to the compounds of formula (Ia) and (Ib), unless otherwise stated.

The compound according to formula (I) may have one CC double bond, which can have either E- or Z-configuration, or being an E-/Z-mixture, if not further specified.

The compound according to formula (I) has one stereo center and exists in the form of two enantiomers. The compound according to formula (I) can be enantiomerically pure, enriched or racemic.

For example, C₁-C₁₁ alkyl can be selected from the group consisting of methyl, ethyl, linear or branched propyl (like n-propyl, iso-propyl,), butyl (like n-butyl, iso-butyl, sec-butyl, tert-butyl etc.), pentyl, hexyl, heptyl, octyl, nonyl, decyl and undecyl.

For example, linear C₇-C₁₀ alkyl can be selected from the group consisting of linear heptyl, octyl, nonyl and decyl.

For example, linear C₇-C₁₀ alkenyl with a terminal CC double bond can be selected from the group consisting of hept-6-en-1-yl, oct-7-en-1-yl, non-8-en-1-yl and dec-9-en-1-yl.

For example, there is provided the use of the compound according to formula (I) as fragrance precursor which is able to release a fragrance with aldehydic notes. In general, the release of fragrant molecules is activated by external trigger, such as hydrolysis, temperature change, oxygen, action of light and/or enzymes.

Some of the compounds according to formula (I) are known from a different context. For example, document WO2013060818A1 describes a process of making a group of compounds with a related structure, which are suitable intermediates or precursor for the preparation of other chemical compounds. The term “intermediates or precursor” means that the compound is not used as such, but is further converted by next preparation steps into a different compound. The document does not refer to fragrance precursors which are able to release a fragrance with aldehydic notes upon activation.

Typically, the compound according to formula (I) is odourless or has low odour, when freshly prepared, and is able to release fragrance, for example having aldehydic fragrance notes. However, in some embodiments of the present invention, the compound according to formula (I) might have an own odor.

By the use of the compound according to formula (I), it is possible to provide the aldehydic fragrance notes over an extended time period and providing a long-lasting release of the aldehydic fragrance notes.

Aldehydic fragrance notes provide an olfactive impression which can usually be described with other words such as: metallic, sharp, piercing (which is more a physical impression or association). Terms also associated with aldehydic are “fatty aldehydic” or “aldehydic green”. The olfactive term aldehydic is not strictly related to the aldehydic chemical feature in the molecule structure. So the typical aldehydic smell became an olfactive reference, independent of the aldehydic chemical feature in the molecular structure.

For example, there is provided the use of the compound according to formula (I) as fragrance precursor wherein the compound is selected from the group consisting of tetradeca-1,13-dien-4-yl acetate, tetradec-1-en-4-yl acetate, tridec-1-en-4-yl acetate, tetradec-1-en-4-yl benzoate, tridec-1-en-4-yl benzoate, tetradeca-1,13-dien-4-yl benzoate, tetradeca-1,13-dien-4-yl 2-naphthoate, tridec-1-en-4-yl 2-naphthoate, tetradec-1-en-4-yl 2-naphthoate, tetradec-1-en-4-yl 2-(naphthalen-1-yl)acetate, tridec-1-en-4-yl 2-(naphthalen-1-yl)acetate, tetradeca-1,13-dien-4-yl 2-(naphthalen-1-yl)acetate, tridec-1-en-4-yl 2-methylundecanoate, tetradec-1-en-4-yl 2-methylundecanoate, tetradec-1-en-4-yl dimethylcarbamate, methyl tetradec-1-en-4-yl carbonate, pentadec-3-en-6-yl acetate, pentadec-3-en-6-yl benzoate, tridec-1-en-4-yl 3,4,5-trimethoxybenzoate, tetradec-1-en-4-yl 3,4,5-trimethoxybenzoate, undec-1-en-4-yl benzoate,dodec-1-en-4-yl benzoate, tetradeca-1,13-dien-4-yl methanesulfonate and tetradeca-1,13-dien-4-yl 4-methylbenzenesulfonate.

According to one aspect of the invention, there is provided the use of the compound according to formula (I) as fragrance precursor, wherein the compound is an ester, and wherein R₁ is selected from the group consisting of linear or branched C₁-C₁₁ alkyl; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; and
R₂, R₃ and R₄ have the same meaning as previously defined.

The compound according to formula (I) described above releases a fragrance compound upon exposure of the precursor compound to ambient air over a long period of time (e.g. several days such as 2-7 days or even longer).

Exposure of the precursor compound to ambient air means exposure to molecular oxygen which might be responsible for the cleavage of the compound according to formula (I) and the release of a fragrance. The concentration of oxygen in the air is sufficient for cleaving the compound according to formula (I) so that the cleavage products can be detected in the ambient air, e.g. by olfaction analysis of headspace samples.

The compounds according to formula (I) are very stable when their exposure to ambient air is limited or prevented, i.e. when stored in neat form in appropriate containers protected from air and light, or when stored in proper solvents, for example in ethanol, isopropanol, diethylene glycol monoethyl ether, glycerol, propylene glycol, 1,2-butylene glycol, dipropylene glycol, isopropyl myristate, triethyl citrate, diethyl phthalate, triacetine and/or diacetine, or when incorporated in consumer products such as detergent, shampoo and fabric conditioner. Thus the compounds according to formula (I) may find use in a broad range of consumer products in which a prolonged and defined release of fragrant compounds is desired.

So in a further aspect of the present invention, there is provided a method to release a fragrance from a compound according to formula (I), wherein said compound is exposed to an environmental trigger. For example, the environmental trigger is ambient air or other oxygen sources.

In some embodiments, stabilizing compounds, for example alpha tocopherol, EDTA, ascorbic acid, BHT, Tinoguard TT, can be added to the compounds according to formula (I), for example in 0.01-1% by weight, to limit or prevent premature cleavage of the compound according to formula (I). In particular, the stabilizing compounds can be used to enhance the stability of the neat compounds according to formula (I).

The compound according to formula (I) may be used alone, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in perfume compositions, for example, carrier materials, and other auxiliary agents commonly used in the art. For example, such known odorant molecules are described in “Perfume and Flavor Chemicals”, S. Arctander, Ed., Vol. I & II, Allured Publishing Corporation, Carol Stream, USA, 2003 and include fragrance compounds of natural or synthetic origin and essential oils.

In a further aspect, the compound according to formula (I) may be used in combination with other fragrance precursors, either with further compounds according to formula (I) or with precursors possessing a different chemical structure. A combination of precursors allows releasing a perfume accord.

In a further aspect, there is provided a fragrance composition comprising at least one compound according to formula (I). For example, the fragrance composition is further comprising one or more known odourant molecules, and/or one or more ingredients or excipients conventionally used in conjunction with odourants in perfume compositions.

The compound according to formula (I) may be comprised in a fragrance composition in widely varying amounts, depending on the effect a perfumer wants to achieve. For example, the compound may be used in 0.01-80% by weight, or in 0.1-50% by weight, or in 1%-20% by weight. These values are given only as examples, and also other amounts might be applied.

As used herein, “carrier material” means a material which is practically neutral from a odourant point of view, i.e. a material that does not significantly alter the organoleptic properties of odourants.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same.

It might also be an ingredient that provides additional benefits such as imparting color or texture. It might also be an ingredient that imparts light resistance or chemical stability to one or more ingredients contained in a perfume composition. A detailed description of the nature and type of adjuvants commonly used in perfume compositions containing same cannot be exhaustive, but it has to be mentioned that said ingredients are well known to a person skilled in the art.

As used herein, “fragrance composition” means any composition comprising the compound according to formula (I) and a base material, e.g. a diluent conventionally used in conjunction with odourants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

The following non limiting list comprises examples of known odourant molecules, which may be combined with the compound according to formula (I) in a fragrance composition:
Essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
Alcohols, e.g. cinnamic alcohol ((E)-3-phenylprop-2-en-1-ol); cis-3-hexenol ((Z)-hex-3-en-1-ol); Citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-_); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
Aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); Hedione® (methyl 3-oxo-2-pentylcyclopentaneacetate); Nympheal (3-(4-isobutyl-2-methylphenyl)propanal); Mahonial (5,9-dimethyl-9-hydroxy-decen-4-al); maltol; methyl cedryl ketone; methylionone; verbenone; and/or vanillin;
Ether and acetals, e.g. Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); rose oxide (4-methyl-2-(2-methylprop-1-en-1-yl)tetrahydro-2H-pyran); and/or Spirambrene® (2′,2′,3,7,7-pentamethylspiro[bicyclo[4.1.0]heptane-2,5′-[1,3]dioxane]);
Esters and lactones, e.g. benzyl acetate; cedryl acetate ((1 S,6R,8aR)-1,4,4,6-tetramethyloctahydro-1H-5,8a-methanoazulen-6-yl acetate); γ-decalactone (6-pentyltetrahydro-2H-pyran-2-one); Helvetolide® (2-(1-(3,3-dimethylcyclohexyl)ethoxy)-2-methylpropyl propionate); γ-undecalactone (5-heptyloxolan-2-one); and/or vetiveryl acetate ((4,8-dimethyl-2-propan-2-ylidene-3,3a,4,5,6,8a-hexahydro-1H-azulen-6-yl) acetate
Macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
Heterocycles, e.g. isobutylquinoline (2-isobutylquinoline).

In a further aspect, there is provided a consumer product comprising at least one compound according to formula (I) and a consumer product base.

For example, the consumer product according to the invention is selected from the group consisting of detergents and cleaning agents, hygiene or care products, preferably in the field of body and hair care, cosmetics and household, preferably from the group consisting of perfume extracts, eau de parfums, eau de toilettes, aftershave lotions, eau de colognes, pre-shave products, splash colognes, perfumed refreshing wipes, acidic, alkaline or neutral detergents, textile fresheners, ironing aids, liquid detergents, powder detergents, laundry pre-treatments, fabric softeners, laundry sheets, washing soaps, washing tablets, dish bar soaps, disinfectants, surface disinfectants, air fresheners, aerosol sprays, waxes and polishes, body care products, hand creams and lotions, foot creams and lotions, depilatory creams and lotions, after-shave creams and lotions, tanning creams and lotions, hair care products (in liquid or solid form), dry shampoo, deodorants, antiperspirants, decorative cosmetic products, candles, lamp oils, incense sticks, insecticides, repellents and fuels.

The consumer product for example is selected from fine fragrance, personal care products (body care products, hair care products, cosmetic products) fabric care products, home care products and air care products. As used herein, “consumer product base” means a composition for use as a consumer product to fulfill specific actions, such as cleaning, softening, and caring or the like.

Personal care products to which the compound of formula (I) can be added include for example all kinds of body care products. Especially interesting products are hair care products, for example shampoos, conditioners and hairsprays, and skin care products, like lotions or creams. Furthermore, the compound of formula (I) may be added to soaps, bath and shower gels and deodorants. The compound of formula (I) can be added to cosmetic products.

Home care products to which the compound of formula (I) can be added include all kinds of detergents, window cleaners, hard surface cleaners, all-purpose cleaners and furniture polishes. Preferably, the products are liquids, e.g. fabric detergent or conditioner compositions. For example, the compounds of formula (I) can act as fragrance precursors in consumer products which further comprise enzymes.

The compound according to formula (I) may be used in a broad range of perfumed consumer products, e.g. in any field of fine and functional perfumery, such as perfumes, air care products, household products, laundry products, body care products and cosmetics. The compound can be employed in widely varying amounts, depending upon the specific article and on the nature and quantity of other odorant ingredients. The proportion of the formula (I) is typically from 0.0001 to 5 weight % of the article. In one embodiment, the compound of formula (I) may be employed in a fabric softener in an amount from 0.001 to 0.3 weight % (e.g. 0.01 to 0.1 including 0.05 weight %). In another embodiment, the compound of formula (I) may be used in fine perfumery but also in consumer products like shampoo, fabric softener or fabric detergents, in amounts from 0.001 to 30 weight % (e.g. up to about 10 or up to 20 weight %), more preferably between 0.01 and 5 weight %. However, these values are given only by way of example, since the experienced perfumer may also achieve effects or may create novel accords with lower or higher concentrations.

In one embodiment there is provided a consumer product comprising an acceptable amount of the compound of formula (I). For example, the fragranced article may comprise 0.000001 weight % to 90 weight % (including 0.00001 weight %; 0.0001 weight %, 0.001 weight %, 0.01 weight %, 0.05 weight %, 0.1 weight %, 0.5 weight %, 1 weight %, 5 weight %, 8 weight %, 10 weight %, 15 weight %, 20 weight %, 25 weight %, 30 weight %, 50 weight %, 60 weight %, 65 weight %) of the compound of formula (I) based on the total amount of the article.

The compound of formula (I) may be employed in a consumer product base simply by directly mixing the compound of the present invention, or a fragrance composition comprising the compound of formula (I), with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base. The consumer product base might further contain entrapment material able to release other fragrant compounds.

Thus, the invention additionally provides a method of manufacturing a consumer product, comprising the incorporation of a compound of formula (I) either by directly admixing it to the consumer product base or by admixing a fragrance composition comprising the compound of formula (I), which may then be mixed with a consumer product base, using conventional techniques and methods. Through the addition of an acceptable amount of the compound of the present invention as hereinabove described, the odor notes of an applied consumer product will be improved, conferred, enhanced, or modified.

Thus, the invention furthermore provides in another aspect a method to confer, enhance, improve or modify the hedonic properties of a fragrance composition or a consumer product, which method comprises adding to said composition or consumer product at least one compound of formula (I).

Some of the compounds according to formula (I) are known from different application, however, most of the compounds are novel.

Therefore, the invention provides a compound according to formula (I)

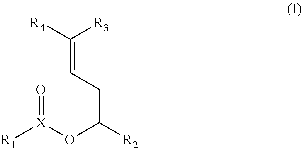

wherein
X is selected from C and S(═O);
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me,
with the proviso, that the compound is not tetradec-1-en-4-yl acetate, tridec-1-en-4-yl acetate, tetradeca-1,13-dien-4-yl acetate, undec-1-en-4-yl benzoate, undec-1-en-4-yl pivalate, and undec-1-en-4-yl acetate, methyl undec-1-en-4-yl carbonate, dodeca-1,11-dien-4-yl acetate, dodec-1-en-4-yl acetate.

For example, there is provided a compound according to formula (Ia)

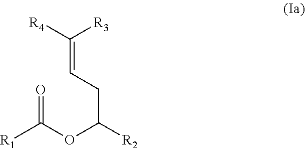

wherein
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me, with the proviso, that the compound is not tetradec-1-en-4-yl acetate, tridec-1-en-4-yl acetate, tetradeca-1,13-dien-4-yl acetate, undec-1-en-4-yl benzoate, undec-1-en-4-yl pivalate, and undec-1-en-4-yl acetate, methyl undec-1-en-4-yl carbonate, dodeca-1,11-dien-4-yl acetate, dodec-1-en-4-yl acetate.

For example, there is provided a compound according to formula (Ia) as defined above, wherein R₂ is selected from the group consisting of linear C₉-C₁₀ alkyl and linear C₇-C₁₀ alkenyl with a terminal CC double bond.

For example, there is provided a compound according to formula (Ib)

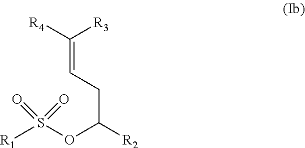

wherein
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me.

The compound according to formula (I) may have one CC double bond, which can have either E- or Z-configuration, or being an E-/Z-mixture, if not further specified.

The compound according to formula (I) has one stereo center and exists in the form of two enantiomers. The compound according to formula (I) can be enantiomerically pure, enriched or racemic.

For example, the compound of formula (I) is selected from the group consisting of tetradec-1-en-4-yl benzoate, tridec-1-en-4-yl benzoate, tetradeca-1,13-dien-4-yl benzoate, tetradeca-1,13-dien-4-yl 2-naphthoate, tridec-1-en-4-yl 2-naphthoate, tetradec-1-en-4-yl 2-naphthoate, tetradec-1-en-4-yl 2-(naphthalen-1-yl)acetate, tridec-1-en-4-yl 2-(naphthalen-1-yl)acetate, tetradeca-1,13-dien-4-yl 2-(naphthalen-1-yl)acetate, tridec-1-en-4-yl 2-methylundecanoate, tetradec-1-en-4-yl 2-methylundecanoate, tetradec-1-en-4-yl dimethylcarbamate, methyl tetradec-1-en-4-yl carbonate, (Z)-pentadec-3-en-6-yl acetate, (Z)-pentadec-3-en-6-yl benzoate, tridec-1-en-4-yl 3,4,5-trimethoxybenzoate, tetradec-1-en-4-yl 3,4,5-trimethoxybenzoate, dodec-1-en-4-yl benzoate, tetradeca-1,13-dien-4-yl methanesulfonate and tetradeca-1,13-dien-4-yl 4-methylbenzenesulfonate.

The compound according to formula (I) may be prepared from an alcohol by esterification with acyl chloride or sulfonyl chloride by methods known in the art.

Alternatively, the compound according to formula (I) may be obtained from a reaction of an aldehyde with allylmagnesium chloride followed by acylation of the resulting alcohol with acyl anhydride or chloride. For example, the compound according to formula (I) may be prepared by a method, wherein the allyl-Grignard addition and ester formation are performed in the same reaction pot, without intermediate isolation or purification. By the Grignard addition a magnesium alkoxide is formed, which is further reacting with the acyl anhydride or chloride, or sulfonyl chloride. In the described one pot reaction, no base is required during acylation or to quench the Grignard between the two steps.

The invention is now further described with reference to the following non-limiting examples. These examples are for the purpose of illustration only and it is understood that variations and modifications can be made by one skilled in the art.

### EXAMPLES

### General:

All reactions were performed under argon using solvents and reagents from commercial suppliers without further purification. Solvents for extraction and chromatography were technical grade and used without further purification. Flash chromatography was performed using Tsingdao Haiyang Chemical silica gel (200-300 mesh) and Santai Technologies silica flash columns. Unless otherwise noted, a mixture of Heptane:MTBE was used as eluent. NMR spectra were recorded with AW 400 MHz Bruker spectrometer instrument. The chemical shifts for ¹H NMR spectra was reported in δ (ppm) referenced to the residual proton signal of the deuterated solvent; coupling constants were expressed in Hertz (Hz). ¹³C NMR spectra were referenced to the carbon signals of the deuterated solvent. The following abbreviations are used: s=singlet, d=doublet, t=triplet, q=quartet, m=multiplet, dd=double doublet, bs=broad singlet. GC/MS spectral data were obtained from an Agilent 6890 N and MSD 5975 using a column HP-5 MS, 30 m, 0.25 mm, 0.25 μm. High resolution mass spectra were determined on a Thermo Fisher Scientific LTQ FT Ultra (ESI-MS) and Waters Micromass GCT Premier (EI-MS).

### Example 1: tetradeca-1,13-dien-4-yl acetate

### Example 1a: tetradeca-1,13-dien-4-ol

A solution of undec-10-enal (42.1 g, 50 mL, 250 mmol) in THF (tetrahydrofuran) (250 mL) was treated slowly at 0° C. with a solution of allylmagnesium chloride in THF (138 mL, 2 M, 276 mmol) and the resulting mixture stirred at 25° C. overnight. The mixture was poured onto iced NH₄Cl solution, extracted 2× with MTBE (methyl tert-butyl ether), washed with brine, dried over MgSO₄ and evaporated. The crude was purified by fractionate distillation over a 5 cm Vigreux column to give tetradeca-1,13-dien-4-ol (38.8 g, 68% yield, 92% purity) as a colorless liquid.

¹H NMR (400 MHz, CDCl3, 298 K) δ (ppm)=5.91-5.75 (m, 2H), 5.19-5.10 (m, 2H), 5.06-4.89 (m, 2H), 3.71-3.59 (m, 1H), 2.36-2.27 (m, 1H), 2.21-2.10 (m, 1H), 2.09-2.00 (m, 2H), 1.71 (d, J=3.9 Hz, 1H), 1.52-1.24 (m, 14H). ¹³C NMR (101 MHz, CDCl3, 298 K) δ (ppm)=139.2, 134.9, 118.0, 114.1, 70.7, 41.9, 36.8, 33.8, 29.6, 29.5, 29.4, 29.1, 28.9, 25.7.

### Example 1b: tetradeca-1,13-dien-4-yl acetate

A solution of tetradeca-1,13-dien-4-ol (5.26 g, 25 mmol) in Toluene (100 mL) was treated with pyridine (5.05 mL, 62.5 mmol) and DMAP (N,N-dimethylpyridin-4-amine) (92 mg, 0.75 mmol) and then treated dropwise, while cooling with a water bath, with a solution of acetyl chloride (1.96 mL, 27.5 mmol) in Toluene (25 mL). The resulting mixture was stirred for 3 h at ambient temperature then poured onto iced 2 N HCl, extracted with MTBE, washed with brine, dried over MgSO₄ and evaporated. The crude was purified by Kugelrohr distillation to give tetradeca-1,13-dien-4-yl acetate (5.38 g, 20 mmol, 92% purity, 78% yield) as a colorless liquid.

¹H NMR (400 MHz, CDCl3, 298 K) δ (ppm)=5.89-5.69 (m, 2H), 5.12-4.87 (m, 5H), 2.38-2.21 (m, 2H), 2.11-1.98 (m, 5H), 1.62-1.49 (m, 2H), 1.43-1.22 (m, 12H). ¹³C NMR (101 MHz, CDCl3, 298 K) δ (ppm)=170.7, 139.1, 133.8, 117.5, 114.1, 73.3, 38.6, 33.8, 33.6, 29.4, 29.4, 29.4, 29.1, 28.9, 25.3, 21.2. MS (EI, 70 eV): 211 (2, [M^{+*}-allyl]), 192 (0), 135 (3), 95 (5), 81 (5), 67 (5), 55 (7), 43 (100).

Odour description (1% solution in EtOH on paper blotter, 24 h): aldehydic, fatty, metallic, green, citrus.

### Example 2: tetradec-1-en-4-yl acetate

A solution of allylmagnesium chloride in THF (16.2 mL, 1.7 molar, 1.1 Eq, 27.5 mmol) was cooled to 0° C. then treated slowly with a solution of undecanal (4.26 g, 5.16 mL, 1 Eq, 25.0 mmol) in THF (5 mL) and the resulting mixture stirred at 0° C. for 2 h. The mixture was then treated slowly with acetic anhydride (3.06 g, 2.83 mL, 1.2 Eq, 30.0 mmol) at 0-15° C. and stirred at 25° C. for 1 h. The mixture was then poured onto 2N HCl (50 mL), extracted with MTBE (2×25 mL), washed with sat. Na₂CO₃ solution (25 mL) and brine (25 mL). The organic layers were dried over MgSO₄, filtered and concentrated to give a crude which was purified by Kugelrohr distillation (oven at 175° C./0.097 mbar) to give tetradec-1-en-4-yl acetate (5.57 g, 21 mmol, 84% yield, 96% purity) as a colorless liquid.

¹H NMR (400 MHz, CDCl3, 298 K) δ (ppm)=5.76 (tdd, J=7.2, 10.1, 17.1 Hz, 1H), 5.15-5.03 (m, 2H), 4.92 (quin, J=6.2 Hz, 1H), 2.37-2.26 (m, 2H), 2.04 (s, 3H), 1.60-1.50 (m, 2H), 1.39-1.20 (m, 16H), 0.93-0.86 (m, 3H). ¹³C NMR (101 MHz, CDCl3, 298 K) δ (ppm)=170.8, 133.8, 117.5, 73.3, 38.6, 33.6, 31.9, 29.6, 29.6, 29.5, 29.4, 29.3, 25.3, 22.7, 21.2, 14.1. MS (EI, 70 eV): 213 (1, [M^{+*}-allyl]), 194 (1), 153 (2), 111 (5), 97 (12), 83 (7), 67 (5), 55 (7), 43 (100), 41 (12).

Odour description (1% solution in EtOH on paper blotter, 24 h): aldehydic, fatty, metallic, green, citrus, mandarin leaf.

### Example 3: tridec-1-en-4-yl acetate

Compound was prepared according to the procedure described in example 2, using decanal (instead of undecanal), to give tridec-1-en-4-yl acetate (62% yield, 89% purity) as a colorless liquid.

¹H NMR (500 MHz, CDCl3, 298 K) δ (ppm)=5.77 (tdd, J=7.1, 10.1, 17.1 Hz, 1H), 5.13-5.04 (m, 2H), 4.93 (quin, J=6.3 Hz, 1H), 2.38-2.27 (m, 2H), 2.05 (s, 3H), 1.59-1.52 (m, 2H), 1.35-1.22 (m, 14H), 0.90 (t, J=7.0 Hz, 3H). ¹³C NMR (126 MHz, CDCl3, 298 K) δ (ppm)=170.8, 133.8, 117.5, 73.4, 38.7, 33.6, 31.9, 29.5, 29.5, 29.3, 25.3, 22.7, 21.2, 14.1. MS (EI, 70 eV): 199 (1, [M^{+*}-allyl]), 180 (1), 157 (1), 139 (3), 97 (5), 83 (9), 67 (4), 55 (6), 43 (100).

Odour description (1% solution in EtOH on paper blotter, 24 h): aldehydic, fatty, metallic, green, citrus.

### Example 4: tetradec-1-en-4-yl benzoate

Compound was prepared according to the procedure described in example 2, using benzoyl chloride (instead of acetic anhydride), to give tetradec-1-en-4-yl benzoate (69% yield) as a colorless liquid.

¹H NMR (400 MHz, CDCl3, 298 K) δ (ppm)=8.10-8.04 (m, 2H), 7.61-7.54 (m, 1H), 7.49-7.43 (m, 2H), 5.85 (tdd, J=7.1, 10.1, 17.1 Hz, 1H), 5.24-5.05 (m, 3H), 2.52-2.43 (m, 2H), 1.79-1.63 (m, 2H), 1.47-1.20 (m, 16H), 0.95-0.86 (m, 3H). ¹³C NMR (101 MHz, CDCl3, 298 K) δ (ppm)=166.2, 133.7, 132.7, 130.8, 129.5, 128.3, 117.7, 74.1, 38.7, 33.7, 31.9, 29.6, 29.6, 29.5, 29.3, 25.3, 22.7, 14.1. MS (EI, 70 eV): 316 (1, [M+*]), 287 (1), 194 (2), 123 (2), 105 (100), 77 (14), 41 (6).

Odour description (1% solution in EtOH on paper blotter, 24 h): aldehydic, fatty, metallic, green, citrus, mandarin leaf.

### Example 5: tridec-1-en-4-yl benzoate

Compound was prepared according to the procedure described in example 2, using decanal (instead of undecanal) and using benzoyl chloride (instead of acetic anhydride), to give tridec-1-en-4-yl benzoate (52% yield) as a colorless liquid.

¹H NMR (400 MHz, CDCl3, 298 K) δ (ppm)=8.07 (dd, J=1.3, 8.4 Hz, 2H), 7.60-7.54 (m, 1H), 7.49-7.43 (m, 2H), 5.86 (tdd, J=7.2, 10.1, 17.1 Hz, 1H), 5.24-5.16 (m, 1H), 5.16-5.05 (m, 2H), 2.51-2.44 (m, 2H), 1.76-1.64 (m, 2H), 1.46-1.21 (m, 14H), 0.93-0.86 (m, 3H). ¹³C NMR (101 MHz, CDCl3, 298 K) δ (ppm)=166.2, 133.7, 132.7, 130.8, 129.5, 128.3, 117.7, 74.1, 38.7, 33.7, 31.9, 29.5, 29.5, 29.3, 25.3, 22.7, 14.1. MS (EI, 70 eV): 302 (1, [M^{+*}]), 261 (1), 180 (1), 123 (1), 105 (100), 77 (15), 41 (7).

Odour description (1% solution in EtOH on paper blotter, 24 h): aldehydic, fatty, green, citrus, mandarin leaf.

### Example 6: tetradeca-1,13-dien-4-yl benzoate

Compound was prepared according to the procedure described in example 1b, using benzoyl chloride (instead of acetyl chloride), to give tetradeca-1,13-dien-4-yl benzoate (77% yield) as a colorless liquid.

¹H NMR (400 MHz, CDCl3, 298 K) δ (ppm)=8.10-8.04 (m, 2H), 7.61-7.54 (m, 1H), 7.50-7.41 (m, 2H), 5.92-5.76 (m, 2H), 5.24-4.91 (m, 5H), 2.50-2.44 (m, 2H), 2.10-2.00 (m, 2H), 1.83-1.62 (m, 2H), 1.52-1.24 (m, 12H). ¹³C NMR (101 MHz, CDCl3, 298 K) δ (ppm)=166.2, 139.2, 133.7, 132.7, 130.7, 129.5, 128.3, 117.7, 114.1, 74.1, 38.7, 33.8, 33.7, 29.5, 29.5, 29.4, 29.1, 28.9, 25.3. MS (EI, 70 eV): 314 (1, [M^{+*}]), 273 (1), 192 (1), 123 (2), 105 (100), 77 (13), 41 (7).

Odour description (1% solution in EtOH on paper blotter, 24 h): aldehydic, fatty, metallic, green, citrus.

### Example 7: tetradeca-1,13-dien-4-yl 2-naphthoate

Compound was prepared according to the procedure described in example 1b, using 2-naphthoyl chloride (instead of acetyl chloride), to give tetradeca-1,13-dien-4-yl benzoate (62% yield) as a yellow liquid.

¹H NMR (400 MHz, CDCl3, 298 K) δ (ppm)=8.63 (d, J=0.7 Hz, 1H), 8.09 (dd, J=1.7, 8.6 Hz, 1H), 7.99 (d, J=7.8 Hz, 1H), 7.95-7.86 (m, 2H), 7.65-7.53 (m, 2H), 5.99-5.72 (m, 2H), 5.34-5.21 (m, 1H), 5.16 (qd, J=1.6, 17.0 Hz, 1H), 5.10 (td, J=1.0, 10.3 Hz, 1H), 5.00 (qd, J=1.8, 17.1 Hz, 1H), 4.94 (tdd, J=1.2, 2.2, 10.1 Hz, 1H), 2.57-2.48 (m, 2H), 2.09-1.98 (m, 2H), 1.86-1.67 (m, 2H), 1.52-1.22 (m, 12H). ¹³C NMR (101 MHz, CDCl3, 298 K) δ (ppm)=166.4, 139.2, 135.5, 133.8, 132.5, 130.9, 129.3, 128.1, 128.1, 128.0, 127.7, 126.6, 125.3, 117.8, 114.1, 74.2, 38.8, 33.8, 33.7, 29.4, 29.1, 28.9, 25.4. MS (EI, 70 eV): 364 (1, [M^{+*}]), 323 (1), 209 (1), 172 (28), 155 (100), 127 (29), 55 (9), 41 (10).

Odour description (1% solution in EtOH on paper blotter, 24 h): aldehydic, green, metallic.

### Example 8: tridec-1-en-4-yl 2-naphthoate

### Example 8a: tridec-1-en-4-ol

Compound was prepared according to the procedure described in example 1a, using decanal (instead of undec-10-enal) to give tridec-1-en-4-ol (95% yield) as a pale yellow liquid.

¹H NMR (400 MHz, CDCl3, 298 K) δ (ppm)=5.85 (dddd, J=6.4, 7.9, 9.6, 17.6 Hz, 1H), 5.22-5.11 (m, 2H), 3.72-3.62 (m, 1H), 2.33 (tddd, J=1.3, 4.2, 6.5, 13.9 Hz, 1H), 2.22-2.09 (m, 1H), 1.62 (br d, J=11.5 Hz, 1H), 1.54-1.23 (m, 16H), 0.95-0.85 (m, 3H). ¹³C NMR (101 MHz, CDCl3, 298 K) δ (ppm)=134.9, 118.0, 70.7, 41.9, 36.8, 31.9, 29.7, 29.6, 29.6, 29.3, 25.7, 22.7, 14.1.

### Example 8b: tridec-1-en-4-yl 2-naphthoate

Compound was prepared according to the procedure described in example 1b, using 2-naphthoyl chloride (instead of acetyl chloride) and tridec-1-en-4-ol (instead of tetradeca-1,13-dien-4-ol), to give tridec-1-en-4-yl 2-naphthoate (75% yield) as a colorless liquid.

¹H NMR (400 MHz, CDCl3, 298 K) δ (ppm)=8.62 (d, J=0.7 Hz, 1H), 8.09 (dd, J=1.6, 8.7 Hz, 1H), 8.03-7.96 (m, 1H), 7.91 (d, J=8.6 Hz, 2H), 7.65-7.52 (m, 2H), 5.90 (tdd, J=7.0, 10.2, 17.1 Hz, 1H), 5.27 (qd, J=5.7, 7.5 Hz, 1H), 5.16 (qd, J=1.6, 17.0 Hz, 1H), 5.10 (tdd, J=1.0, 2.0, 10.0 Hz, 1H), 2.57-2.48 (m, 2H), 1.86-1.67 (m, 2H), 1.52-1.20 (m, 14H), 0.94-0.84 (m, 3H). ¹³C NMR (101 MHz, CDCl3, 298 K) δ (ppm)=166.4, 135.5, 133.8, 132.5, 130.9, 129.3, 128.1, 128.1, 128.0, 127.7, 126.6, 125.3, 117.8, 74.2, 38.8, 33.7, 31.9, 29.5, 29.5, 29.3, 25.4, 22.7, 14.1. MS (EI, 70 eV): 352 (1, [M^{+*}]), 337 (1), 311 (1), 172 (26), 155 (100), 127 (31), 55 (5), 41 (9).

Odour description (1% solution in EtOH on paper blotter, 24 h): aldehydic, green, metallic, mandarin leaf.

### Example 9: tetradec-1-en-4-yl 2-naphthoate

### Example 9a: tetradec-1-en-4-ol

Compound was prepared according to the procedure described in example 1a, using undecanal (instead of undec-10-enal) to give tetradec-1-en-4-ol (96% yield) as a colorless liquid.

¹H NMR (400 MHz, CDCl3, 298 K) δ (ppm)=5.92-5.78 (m, 1H), 5.22-5.09 (m, 2H), 3.66 (br s, 1H), 2.32 (tddd, J=1.3, 4.2, 6.6, 13.9 Hz, 1H), 2.21-2.10 (m, 1H), 1.64 (s, 1H), 1.54-1.22 (m, 18H), 0.95-0.83 (m, 3H). ¹³C NMR (101 MHz, CDCl3, 298 K) δ (ppm)=134.9, 118.0, 70.7, 41.9, 36.8, 31.9, 29.7, 29.6, 29.6, 29.3, 25.7, 25.6, 22.7, 14.1.

### Example 9b: tetradec-1-en-4-yl 2-naphthoate

Compound was prepared according to the procedure described in example 1b, using 2-naphthoyl chloride (instead of acetyl chloride) and tetradec-1-en-4-ol (instead of tetradeca-1,13-dien-4-ol), to give tetradec-1-en-4-yl 2-naphthoate (66% yield) as a colorless liquid.

¹H NMR (400 MHz, CDCl3, 298 K) δ (ppm)=8.62 (d, J=0.7 Hz, 1H), 8.09 (dd, J=1.7, 8.6 Hz, 1H), 8.03-7.96 (m, 1H), 7.91 (d, J=8.6 Hz, 2H), 7.66-7.53 (m, 2H), 5.90 (tdd, J=7.1, 10.2, 17.2 Hz, 1H), 5.27 (qd, J=5.9, 7.4 Hz, 1H), 5.20-5.06 (m, 2H), 2.57-2.49 (m, 2H), 1.86-1.67 (m, 2H), 1.52-1.19 (m, 16H), 0.94-0.84 (m, 3H). ¹³C NMR (101 MHz, CDCl3, 298 K) δ (ppm)=166.4, 135.5, 133.8, 132.5, 130.9, 129.3, 128.1, 128.1, 128.0, 127.7, 126.6, 125.3, 117.8, 77.2, 74.2, 38.8, 33.7, 31.9, 29.6, 29.6, 29.5, 29.3, 25.4, 22.7, 14.1. MS (EI, 70 eV): 366 (1, [M^{+*}]), 325 (1), 194 (2), 172 (27), 155 (100), 127 (29), 55 (5), 41 (9).

Odour description (1% solution in EtOH on paper blotter, 24 h): green, metallic, aldehydic.

### Example 10: tetradec-1-en-4-yl 2-(naphthalen-1-yl)acetate

Compound was prepared according to the procedure described in example 1b, using 2-(naphthalen-1-yl)acetyl chloride (instead of acetyl chloride) and tetradec-1-en-4-ol (instead of tetradeca-1,13-dien-4-ol), to give tetradec-1-en-4-yl 2-naphthoate (76% yield) as a colorless liquid.

¹H NMR (400 MHz, CDCl3, 298 K) δ (ppm)=8.08-8.00 (m, 1H), 7.92-7.86 (m, 1H), 7.85-7.79 (m, 1H), 7.58-7.48 (m, 2H), 7.48-7.41 (m, 2H), 5.73-5.60 (m, 1H), 5.03-4.90 (m, 3H), 4.08 (s, 2H), 2.32-2.24 (m, 2H), 1.56-1.46 (m, 2H), 1.38-1.09 (m, 16H), 0.97-0.89 (m, 3H). ¹³C NMR (101 MHz, CDCl3, 298 K) δ (ppm)=171.3, 133.8, 133.6, 132.1, 130.9, 128.7, 128.0, 127.9, 126.2, 125.7, 125.4, 124.0, 117.5, 73.9, 39.6, 38.6, 33.5, 31.9, 29.6, 29.5, 29.5, 29.4, 29.4, 25.1, 22.7, 14.1. MS (EI, 70 eV): 380 (15, [M^{+*}]), 339 (1), 186 (19), 169 (3), 141 (100), 115 (13).

Odour description (1% solution in EtOH on paper blotter, 24 h): aldehydic, green, metallic, fatty, citrus leaf, hot iron.

### Example 11: tridec-1-en-4-yl 2-(naphthalen-1-yl)acetate

Compound was prepared according to the procedure described in example 1b, using 2-(naphthalen-1-yl)acetyl chloride (instead of acetyl chloride) and tridec-1-en-4-ol (instead of tetradeca-1,13-dien-4-ol), to give tridec-1-en-4-yl 2-(naphthalen-1-yl)acetate (67% yield) as a pale yellow liquid.

¹H NMR (400 MHz, CDCl3, 298 K) δ (ppm)=8.08-8.01 (m, 1H), 7.92-7.87 (m, 1H), 7.85-7.78 (m, 1H), 7.58-7.49 (m, 2H), 7.48-7.42 (m, 2H), 5.74-5.61 (m, 1H), 5.05-4.90 (m, 3H), 4.08 (s, 2H), 2.35-2.22 (m, 2H), 1.57-1.45 (m, 2H), 1.40-1.10 (m, 14H), 0.93 (t, J=7.0 Hz, 3H). ¹³C NMR (101 MHz, CDCl3, 298 K) δ (ppm)=171.3, 133.8, 133.6, 132.1, 130.9, 128.7, 128.0, 127.9, 126.2, 125.7, 125.4, 124.0, 117.6, 73.9, 39.6, 38.6, 33.6, 31.9, 29.5, 29.5, 29.4, 29.3, 25.1, 22.7, 14.2. MS (EI, 70 eV): 366 (7, [M^{+*}]), 325 (1), 186 (16), 169 (3), 141 (100), 115 (14), 41 (14).

Odour description (1% solution in EtOH on paper blotter, 24 h): green, citrus, aldehydic, metallic, fatty, citrus leaf, hot iron.

### Example 12: tetradeca-1,13-dien-4-yl 2-(naphthalen-1-yl)acetate

Compound was prepared according to the procedure described in example 1b, using 2-(naphthalen-1-yl)acetyl chloride (instead of acetyl chloride), to give tetradeca-1,13-dien-4-yl 2-(naphthalen-1-yl)acetate (37% yield) as a pale yellow liquid.

¹H NMR (400 MHz, CDCl3, 298 K) δ (ppm)=8.07-8.01 (m, 1H), 7.92-7.86 (m, 1H), 7.81 (dd, J=2.8, 6.5 Hz, 1H), 7.58-7.48 (m, 2H), 7.48-7.42 (m, 2H), 5.85 (tdd, J=6.6, 10.3, 17.1 Hz, 1H), 5.73-5.60 (m, 1H), 5.08-4.89 (m, 5H), 4.08 (s, 2H), 2.32-2.23 (m, 2H), 2.12-2.01 (m, 2H), 1.56-1.44 (m, 2H), 1.44-1.08 (m, 12H). ¹³C NMR (101 MHz, CDCl3, 298 K) δ (ppm)=171.3, 139.2, 133.6, 132.1, 130.9, 128.7, 128.0, 127.9, 126.2, 125.7, 125.4, 124.0, 117.6, 114.1, 73.9, 39.6, 38.6, 33.8, 33.5, 29.4, 29.3, 29.1, 28.9, 25.1. MS (EI, 70 eV): 378 (7, [M^{+*}]), 337 (2), 186 (19), 169 (2), 141 (100), 115 (14), 55 (16).

Odour description (1% solution in EtOH on paper blotter, 24 h): aldehydic, metallic, citrus leaf, hot iron, green.

### Example 13: tridec-1-en-4-yl 2-methylundecanoate

Example 13a: 2-methylundecanoyl chloride A mixture of 2-methylundecanoic acid (6.92 g, 1 Eq, 34.5 mmol) and DMF (N,N-dimethylformamide) (37.9 mg, 40.1 μL, 0.015 Eq, 518 μmol) was treated dropwise at 10° C. with thionyl chloride (14.4 g, 8.78 mL, 3.5 Eq, 121 mmol) and stirred at room temperature for 2 h. The resulting mixture was directly purified by Kugelrohr distillation to give 2-methylundecanoyl chloride (6.35 g, 33 mmol 98% purity, 95% yield) as a colorless liquid.

¹H NMR (500 MHz, CDCl3, 298 K) δ (ppm)=2.88 (sxt, J=6.8 Hz, 1H), 1.88-1.76 (m, 1H), 1.58-1.48 (m, 1H), 1.41-1.21 (m, 17H), 0.95-0.86 (m, 3H). ¹³C NMR (126 MHz, CDCl3, 298 K) δ (ppm)=177.8, 51.5, 33.4, 31.9, 29.5, 29.4, 29.4, 29.3, 26.7, 22.7, 17.0, 14.1.

Example 13b: tridec-1-en-4-yl 2-methylundecanoate Compound was prepared according to the procedure described in example 1b, using 2-methylundecanoyl chloride (instead of acetyl chloride) and tridec-1-en-4-ol (instead of tetradeca-1,13-dien-4-ol), to give tridec-1-en-4-yl 2-methylundecanoate (68% yield) as a colorless liquid (1:1 mixture of diastereomers).

¹H NMR (500 MHz, CDCl3, 298 K, 1:1 mixture of diastereomers) δ (ppm)=5.82-5.70 (m, 1H), 5.11-5.04 (m, 2H), 4.97-4.90 (m, 1H), 2.46-2.37 (m, 1H), 2.37-2.26 (m, 2H), 1.71-1.61 (m, 1H), 1.60-1.51 (m, 2H), 1.44-1.36 (m, 1H), 1.36-1.21 (m, 28H), 1.17-1.12 (m, 3H), 0.93-0.86 (m, 6H). ¹³C NMR (126 MHz, CDCl3, 298 K, 1:1 mixture of diastereomers) δ (ppm)=176.6, 176.5, 133.9, 117.4, 117.4, 72.8, 72.7, 40.0, 39.9, 38.8, 38.8, 33.9, 33.6, 31.9, 29.6, 29.6, 29.5, 29.4, 27.3, 25.3, 25.3, 22.7, 17.3, 17.3, 14.1. MS (EI, 70 eV): 380 (1, [M^{+*}]), 323 (1), 309 (1), 295 (1), 281 (1), 267 (1), 254 (1), 225 (1), 201 (4), 183 (84), 180 (11), 155 (18), 113 (13), 99 (31), 85 (58), 71 (57), 57 (100), 43 (64).

Odour description (1% solution in EtOH on paper blotter, 24 h): aldehydic, metallic, balsamic, incense, citrus, green.

### Example 14: tetradec-1-en-4-yl 2-methylundecanoate

Compound was prepared according to the procedure described in example 1b, using 2-methylundecanoyl chloride (instead of acetyl chloride) and tetradec-1-en-4-ol (instead of tetradeca-1,13-dien-4-ol), to give tetradec-1-en-4-yl 2-methylundecanoate (64% yield) as a colorless liquid (1:1 mixture of diastereomers).

¹H NMR (500 MHz, CDCl3, 298 K, 1:1 mixture of diastereomers) δ (ppm)=5.82-5.71 (m, 1H), 5.12-5.03 (m, 2H), 4.97-4.89 (m, 1H), 2.47-2.37 (m, 1H), 2.37-2.26 (m, 2H), 1.71-1.63 (m, 1H), 1.59-1.51 (m, 2H), 1.44-1.37 (m, 1H), 1.36-1.22 (m, 30H), 1.17-1.12 (m, 3H), 0.92-0.87 (m, 6H). ¹³C NMR (126 MHz, CDCl3, 298 K, 1:1 mixture of diastereomers) δ (ppm)=176.6, 176.5, 133.9, 117.5, 117.5, 72.8, 72.7, 40.0, 39.9, 38.8, 38.8, 33.9, 33.6, 31.9, 29.6, 29.6, 29.6, 29.6, 29.5, 29.4, 27.3, 25.3, 25.3, 22.7, 17.4, 17.3, 14.1. MS (EI, 70 eV): 394 (1, [M^{+*}]), 194 (11), 183 (98), 155 (17), 113 (13), 99 (31), 85 (60), 71 (58), 57 (100), 43 (66).

Odour description (1% solution in EtOH on paper blotter, 24 h): aldehydic, metallic, balsamic, incense, citrus, green.

### Example 15: tetradec-1-en-4-yl dimethylcarbamate

A flask containing NaH (55% dispersion in mineral oil) (0.24 g, 5.5 mmol) and Toluene (2.5 mL) treated slowly at 25° C. with a solution of tetradec-1-en-4-ol (1.1 g, 5.0 mmol) in Toluene (2.5 mL) and the resulting mixture heated to reflux for 1 h. The resulting mixture was cooled to 0° C. and treated slowly with a solution of dimethylcarbamic chloride (0.55 mL, 0.65 g, 6.0 mmol) in Toluene (1.5 mL) and the mixture allowed to stir at 25° C. for 16 h. The mixture was then poured onto water, extracted 2× with MTBE, washed with brine, dried over MgSO₄ and the resulting crude material purified by Kugelrohr distillation to give tetradec-1-en-4-yl dimethylcarbamate (1.32 g, 90% purity, 84% yield) as a colorless liquid.

¹H NMR (400 MHz, CDCl3, 298 K) δ (ppm)=5.79 (tdd, J=7.1, 10.1, 17.1 Hz, 1H), 5.12-5.01 (m, 2H), 4.79 (quin, J=6.1 Hz, 1H), 2.91 (br s, 6H), 2.37-2.29 (m, 2H), 1.61-1.51 (m, 2H), 1.39-1.22 (m, 16H), 0.93-0.86 (m, 3H). ¹³C NMR (101 MHz, CDCl3, 298 K) δ (ppm)=156.5, 134.2, 117.2, 74.3, 38.9, 36.3 (br), 35.7 (br), 33.8, 31.9, 29.6, 29.3, 25.3, 22.7, 14.1. MS (EI, 70 eV): 283 (1, [M^{+*}]), 242 (1), 194 (3), 90 (16), 72 (100), 41 (13).

Odour description (1% solution in EtOH on paper blotter, 24 h): powdery, aldehydic, fatty, metallic, citrus, green.

### Example 16: methyl tetradec-1-en-4-yl carbonate

Compound was prepared according to the procedure described in example 1b, using methyl chloroformate (instead of acetyl chloride) and tetradec-1-en-4-ol (instead of tetradeca-1,13-dien-4-ol), to give methyl tetradec-1-en-4-yl carbonate (87% yield) as a colorless liquid.

¹H NMR (400 MHz, CDCl3, 298 K) 6 (ppm)=5.79 (tdd, J=7.1, 10.2, 17.2 Hz, 1H), 5.15-5.06 (m, 2H), 4.80-4.70 (m, 1H), 3.78 (s, 3H), 2.41-2.34 (m, 2H), 1.70-1.52 (m, 2H), 1.42-1.21 (m, 16H), 0.93-0.86 (m, 3H). ¹³C NMR (101 MHz, CDCl3, 298 K) δ (ppm)=155.6, 133.3, 117.9, 77.9, 54.5, 38.5, 33.5, 31.9, 29.5, 29.3, 25.1, 22.7, 14.1. MS (EI, 70 eV): 194 (4, [M^{+*}-HOC(O)OCH₃]), 153 (4), 111 (28), 97 (100), 83 (85), 77 (14).

Odour description (1% solution in EtOH on paper blotter, 24 h): aldehydic, fatty, metallic, green, marine.

### Example 17: (Z)-pentadec-3-en-6-yl acetate

Example 17a: (Z)-pentadec-3-en-6-ol A flask was charged with magnesium (495 mg, 1 Eq, 20.4 mmol) and 0.1 g bromononane and the Grignard formation was started with a heating gun. Over 25 min a solution of 1-bromononane (4.22 g, 1 Eq, 20.4 mmol) in THF (20 mL) was added dropwise. After the addition the reaction mixture was heated to 50° C. for 30 min until all magnesium turnings were dissolved. The Grignard reagent was cooled to −20° C. and treated dropwise over 10 min with a solution of freshly distilled (Z)-hex-3-enal (2.00 g, 1 Eq, 20.4 mmol) in THF (10 mL). After the addition the cooling bath was removed and the reaction mixture was stirred for 1 h at ambient temp. The reaction mixture was poured into sat. NH₄Cl-solution and was extracted 2× with MTBE, washed with water and brine until neutral, dried over MgSO4, filtered and concentrated. The resulting material was purified by chromatography on silica gel (eluting with MTBE in heptane) followed by Kugelrohr distillation to give (Z)-pentadec-3-en-6-ol (1.4 g, 98% purity, 30% yield) as a colorless oil.

¹H NMR (400 MHz, CDCl3, 298 K) 6 (ppm)=5.64-5.55 (m, 1H), 5.46-5.35 (m, 1H), 3.69-3.56 (m, 1H), 2.27-2.20 (m, 2H), 2.15-2.04 (m, 2H), 1.56 (br s, 1H), 1.53-1.41 (m, 2H), 1.38-1.23 (m, 14H), 0.99 (t, J=7.5 Hz, 3H), 0.94-0.87 (m, 3H). ¹³C NMR (101 MHz, CDCl3, 298 K) 6 (ppm)=135.1, 124.6, 71.5, 36.9, 35.2, 31.9, 29.7, 29.6, 29.6, 29.3, 25.8, 22.7, 20.7, 14.3, 14.1.

### Example 17b: (Z)-pentadec-3-en-6-yl acetate

Compound was prepared according to the procedure described in example 1b, using (Z)-pentadec-3-en-6-ol (instead of tetradeca-1,13-dien-4-ol), to give (Z)-pentadec-3-en-6-yl acetate (67% yield) as a colorless liquid.

¹H NMR (400 MHz, CDCl3, 298 K) δ (ppm)=5.55-5.46 (m, 1H), 5.38-5.27 (m, 1H), 4.89 (quin, J=6.2 Hz, 1H), 2.37-2.22 (m, 2H), 2.11-2.01 (m, 4H), 1.59-1.51 (m, 2H), 1.37-1.23 (m, 14H), 0.98 (t, J=7.6 Hz, 3H), 0.93-0.87 (m, 3H). ¹³C NMR (101 MHz, CDCl3, 298 K) δ (ppm)=170.8, 134.3, 123.6, 74.0, 33.6, 31.9, 31.8, 29.5, 29.5, 29.3, 25.4, 22.7, 21.3, 20.6, 14.2, 14.1. MS (EI, 70 eV): 208 (7, [M^{+*}-HOC(O)CH₃]), 199 (1), 95 (10), 82 (24), 43 (100).

Odour description (1% solution in EtOH on paper blotter, 24 h): green, fatty, watery, metallic, aldehydic, mandarin.

### Example 18: (Z)-pentadec-3-en-6-yl benzoate

Compound was prepared according to the procedure described in example 1b, using benzoyl chloride (instead of acetyl chloride) and (Z)-pentadec-3-en-6-ol (instead of tetradeca-1,13-dien-4-ol), to give (Z)-pentadec-3-en-6-yl benzoate (41% yield) as a colorless liquid.

¹H NMR (400 MHz, CDCl3, 298 K) δ (ppm)=8.10-8.03 (m, 2H), 7.62-7.53 (m, 1H), 7.50-7.43 (m, 2H), 5.55-5.36 (m, 2H), 5.21-5.11 (m, 1H), 2.55-2.36 (m, 2H), 2.15-1.95 (m, 2H), 1.79-1.62 (m, 2H), 1.49-1.19 (m, 14H), 0.96 (t, J=7.6 Hz, 3H), 0.92-0.86 (m, 3H). ¹³C NMR (101 MHz, CDCl3, 298 K) δ (ppm)=166.3, 134.4, 132.7, 130.8, 129.5, 129.5, 128.3, 123.5, 74.7, 33.7, 31.9, 31.9, 29.5, 29.3, 25.4, 22.7, 20.7, 14.1, 14.1. MS (EI, 70 eV): 261 (1), 208 (7), 105 (100), 77 (16).

Odour description (1% solution in EtOH on paper blotter, 24 h): green, metallic, aldehydic, mandarin.

### Example 19: tridec-1-en-4-yl 3,4,5-trimethoxybenzoate

Compound was prepared according to the procedure described in example 1b, using 3,4,5-trimethoxybenzoyl chloride (instead of acetyl chloride) and tridec-1-en-4-ol (instead of tetradeca-1,13-dien-4-ol), to give tridec-1-en-4-yl 3,4,5-trimethoxybenzoate (63% yield) as a colorless liquid.

¹H NMR (400 MHz, CDCl3, 298 K) δ (ppm)=7.31 (s, 2H), 5.84 (tdd, J=7.1, 10.2, 17.2 Hz, 1H), 5.21-5.05 (m, 3H), 3.92 (s, 6H), 3.91 (s, 3H), 2.50-2.42 (m, 2H), 1.81-1.61 (m, 2H), 1.49-1.21 (m, 14H), 0.92-0.84 (m, 3H). ¹³C NMR (101 MHz, CDCl3, 298 K) δ (ppm)=165.8, 152.9, 142.1, 133.8, 125.8, 117.7, 106.8, 74.3, 60.9, 56.2, 38.7, 33.7, 31.9, 29.5, 29.5, 29.3, 25.3, 22.7, 14.1. MS (EI, 70 eV): 392 (11, [M^{+*}]), 351 (1), 212 (51), 197 (16), 195 (100), 41 (18).

Odour description (1% solution in EtOH on paper blotter, 24 h): aldehydic, fatty, green, metallic, vibrant, citrus, mandarin leaf.

### Example 20: tetradec-1-en-4-yl 3,4,5-trimethoxybenzoate

Compound was prepared according to the procedure described in example 1b, using 3,4,5-trimethoxybenzoyl chloride (instead of acetyl chloride) and tetradec-1-en-4-ol (instead of tetradeca-1,13-dien-4-ol), to give tetradec-1-en-4-yl 3,4,5-trimethoxybenzoate (72% yield) as a colorless liquid.

¹H NMR (400 MHz, CDCl3, 298 K) δ (ppm)=7.31 (s, 2H), 5.91-5.77 (m, 1H), 5.21-5.05 (m, 3H), 3.92 (s, 6H), 3.92 (s, 3H), 2.50-2.42 (m, 2H), 1.79-1.61 (m, 2H), 1.50-1.22 (m, 16H), 0.92-0.84 (m, 3H). ¹³C NMR (101 MHz, CDCl3, 298 K) δ (ppm)=165.9, 152.9, 142.1, 133.8, 125.8, 117.7, 106.8, 74.3, 60.9, 56.2, 38.7, 33.7, 31.9, 29.6, 29.5, 29.3, 25.4, 22.7, 14.1. MS (EI, 70 eV): 406 (11, [M^{+*}]), 365 (1), 212 (53), 195 (100).

Odour description (1% solution in EtOH on paper blotter, 24 h): green, aldehydic, fatty, metallic, citrus, mandarin leaf.

### Example 21: undec-1-en-4-yl benzoate

### Example 21a: undec-1-en-4-ol

Compound was prepared according to the procedure described in example 1a, using octanal (instead of undec-10-enal) to give undec-1-en-4-ol (98% yield) as a pale yellow liquid.

¹H NMR (400 MHz, CDCl3, 298 K) δ (ppm)=5.92-5.77 (m, 1H), 5.20-5.10 (m, 2H), 3.70-3.61 (m, 1H), 2.32 (tddd, J=1.3, 4.1, 6.6, 13.9 Hz, 1H), 2.21-2.09 (m, 1H), 1.67 (br s, 1H), 1.53-1.24 (m, 12H), 0.95-0.85 (m, 3H). ¹³C NMR (101 MHz, CDCl3, 298 K) δ (ppm)=134.9, 118.0, 70.7, 41.9, 36.8, 31.8, 29.6, 29.3, 25.7, 22.7, 14.1.

### Example 21b: undec-1-en-4-yl benzoate

Compound was prepared according to the procedure described in example 1b, using benzoyl chloride (instead of acetyl chloride), and undec-1-en-4-ol (instead of tetradeca-1,13-dien-4-ol) to give undec-1-en-4-yl benzoate (61% yield) as a colorless liquid.

¹H NMR (400 MHz, CDCl3, 298 K) δ (ppm)=8.11-8.03 (m, 2H), 7.61-7.54 (m, 1H), 7.51-7.42 (m, 2H), 5.85 (tdd, J=7.0, 10.1, 17.1 Hz, 1H), 5.24-5.16 (m, 1H), 5.16-5.06 (m, 2H), 2.52-2.42 (m, 2H), 1.78-1.64 (m, 2H), 1.50-1.20 (m, 10H), 0.94-0.85 (m, 3H). ¹³C NMR (101 MHz, CDCl3, 298 K) δ (ppm)=166.2, 133.7, 132.7, 130.7, 129.5, 128.3, 117.7, 74.1, 38.7, 33.7, 31.8, 29.5, 29.2, 25.3, 22.6, 14.1.

Odour description (1% solution in EtOH on paper blotter, 24 h): green, coriander, aldehydic, metallic, fatty, birch leaf.

### Example 22: dodec-1-en-4-yl benzoate

### Example 22a: dodec-1-en-4-ol

Compound was prepared according to the procedure described in example 1a, using nonanal (instead of undec-10-enal) to give dodec-1-en-4-ol (99% yield) as a pale yellow liquid. 10 ¹H NMR (400 MHz, CDCl3, 298 K) δ (ppm)=5.92-5.77 (m, 1H), 5.20-5.09 (m, 2H), 3.70-3.60 (m, 1H), 2.31 (tddd, J=1.3, 4.2, 6.6, 13.9 Hz, 1H), 2.20-2.09 (m, 1H), 1.69 (d, J=3.7 Hz, 1H), 1.53-1.22 (m, 14H), 0.94-0.85 (m, 3H). ¹³C NMR (101 MHz, CDCl3, 298 K) δ (ppm)=134.9, 118.0, 70.7, 41.9, 36.8, 31.9, 29.7, 29.6, 29.3, 25.7, 22.7, 14.1.

### Example 22b: dodec-1-en-4-yl benzoate

Compound was prepared according to the procedure described in example 1b, using benzoyl chloride (instead of acetyl chloride), and dodec-1-en-4-ol (instead of tetradeca-1,13-dien-4-ol) to give dodec-1-en-4-yl benzoate (51% yield) as a colorless liquid.

¹H NMR (400 MHz, CDCl3, 298 K) δ (ppm)=8.09-8.04 (m, 2H), 7.61-7.54 (m, 1H), 7.49-7.43 (m, 2H), 5.85 (tdd, J=7.1, 10.2, 17.2 Hz, 1H), 5.24-5.16 (m, 1H), 5.16-5.05 (m, 2H), 2.53-2.43 (m, 2H), 1.80-1.62 (m, 2H), 1.48-1.20 (m, 12H), 0.94-0.84 (m, 3H). ¹³C NMR (101 MHz, CDCl3, 298 K) δ (ppm)=166.2, 133.7, 132.7, 129.5, 128.3, 117.7, 74.1, 38.7, 33.7, 31.8, 29.5, 29.5, 29.2, 25.3, 22.7, 14.1.

Odour description (1% solution in EtOH on paper blotter, 24 h): aldehydic, metallic, fatty, citrus, mandarin leaf.

### Example 23: Application in liquid detergent

### a) Sample Preparation

0.2% by weight of a compound according to formula (I) was incorporated into unperfumed liquid as odorant detergent base by magnetic stirring at room temperature for 24 h.

### b) Wash Test and Sensory Evaluation

A 40° C. machine wash cycle was performed using 55 g of the above prepared liquid detergent sample and odor-neutral cotton/elastan mixed fabric T-shirts. The wet and line-dried fabric (1 and 4 days) was assessed by a panel of 4-6 experts with regard to odor intensity and quality. The odor intensity was recorded on an intensity scale of 0 (odorless) to 5 (extremely strong).

TABLE 1

| Sample | wet | 1 day | 4 days | Odor |
|---|---|---|---|---|
| tetradeca-1,13-dien-4-yl | 1.3 | 3.1 | 3.8 | fresh aldehydic, |
| acetate (example 1) | | | | metallic, hot iron |
| tetradec-1-en-4-yl acetate | 1.1 | 2.1 | 3.2 | mandarin, |
| (example 2) | | | | aldehydic, fresh |
| methyl tetradec-1-en-4-yl | 0.8 | 2.0 | 3.5 | aldehydic, |
| carbonate (example 16) | | | | mandarin, floral |
| tetradec-1-en-4-yl | 1.5 | 2.2 | 3.0 | green, mandarin, |
| dimethylcarbamate | | | | aldehydic |
| (example 15) | | | | |
| tetradeca-1,13-dien-4-yl | 1.5 | 2.3 | 3.4 | aldehydic, green, |
| benzoate (example 6) | | | | mandarin, hot |
| | | | | iron |
| tetradeca-1,13-dien-4-yl | 1.4 | 2.8 | 4.1 | mandarin, |
| methanesulfonate | | | | aldehydic, fresh, |
| (example 24) | | | | citrus, hot iron |

As can be seen from Table 1, all materials tested exhibited little odor in wet stage and released fresh aldehydic notes over the course of the subsequent days resulting in strong odor impressions (score>3) on dried fabric, even increasing in time. The smell on wet stage was always non-specific, associated with the blank smell of fabric washed with an unperfumed detergent.

### Example 24: tetradeca-1,13-dien-4-yl methanesulfonate

A flask was charged with tetradeca-1,13-dien-4-ol (3.00 g, 14.3 mmol), Triethylamine (1.73 g, 2.39 mL, 1.2 Eq, 17.1 mmol) and dichloromethane (DCM) (10 mL) and the mixture cooled to 5° C. The mixture was then treated dropwise at 5° C. with a solution of methanesulfonyl chloride (1.96 g, 1.32 mL, 1.2 Eq, 17.1 mmol) in DCM (4 mL). After the addition the cooling bath was removed and the yellow suspension was stirred at ambient temperature for 30 min. The reaction mixture was then poured into iced 1M aqueous HCl-solution (100 mL) and extracted with MTBE (2×100 mL), the organic layers were washed with water and brine until neutral, dried over MgSO₄, filtered and concentrated. The crude material was purified by chromatography on silica gel to give tetradeca-1,13-dien-4-yl methanesulfonate (2.10 g, 51% yield) as a colorless liquid.

¹H NMR (400 MHz, CDCl3, 298 K) δ (ppm)=5.91-5.73 (m, 2H), 5.22-5.14 (m, 2H), 5.05-4.92 (m, 2H), 4.75 (quin, J=6.1 Hz, 1H), 3.01 (s, 3H), 2.56-2.41 (m, 2H), 2.12-2.00 (m, 2H), 1.79-1.63 (m, 2H), 1.50-1.24 (m, 12H). ¹³C NMR (101 MHz, CDCl3, 298 K) δ (ppm)=139.2, 132.6, 118.9, 114.1, 82.9, 39.0, 38.8, 34.2, 33.8, 29.4, 29.4, 29.3, 29.1, 28.9, 25.0. MS (EI, 70 eV): 209 (1, [M-CH₃SO₂]+*), 192 (1), 151 (7), 135 (10), 121 (11), 109 (34), 95 (80), 81 (57), 67 (64), 55 (100), 41 (64).

Odour description (1% solution in EtOH on paper blotter, 24 h): aldehydic, fresh, citrus, mandarin, hot iron, metallic, coriander.

### Example 25: tetradeca-1,13-dien-4-yl 4-methylbenzenesulfonate

Prepared according to the procedure described in Example 24, replacing methanesulfonyl chloride with p-toluenesulfonyl chloride (3.26 g, 1.2 Eq, 17.1 mmol), to give tetradeca-1,13-dien-4-yl 4-methylbenzenesulfonate (2.20 g, 42% yield) as a colorless liquid.

¹H NMR (400 MHz, CDCl3, 298 K) δ (ppm)=7.85-7.78 (m, 2H), 7.35 (dd, J=0.7, 8.6 Hz, 2H), 5.83 (tdd, J=6.6, 10.3, 17.1 Hz, 1H), 5.73-5.59 (m, 1H), 5.09-4.92 (m, 4H), 4.58 (quin, J=6.1 Hz, 1H), 2.46 (s, 3H), 2.38 (tddd, J=1.3, 2.8, 5.8, 7.2 Hz, 2H), 2.09-2.01 (m, 2H), 1.64-1.53 (m, 2H), 1.43-1.33 (m, 2H), 1.33-1.12 (m, 12H). ¹³C NMR (101 MHz, CDCl3, 298 K) δ (ppm)=144.4, 139.2, 134.6, 132.3, 129.6, 127.8, 118.6, 114.2, 83.1, 38.8, 33.8, 33.6, 29.3, 29.2, 29.1, 28.9, 24.7, 21.6.

Odour description (1% solution in EtOH on paper blotter, 24 h): aldehydic, hot iron, citrus, metallic.

1. A method of using a fragrance precursor which is a compound according to formula (I) to release a fragrance, the method comprising activating the compound of formula (I) thereby releasing a fragrance, wherein the compound of formula (I) is 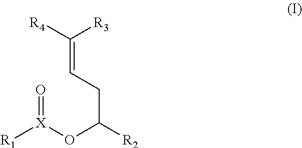
wherein
X is selected from C and S(═O);
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me.

2. The method according to claim 1, wherein the compound of formula (I) is the compound according to formula (Ia) 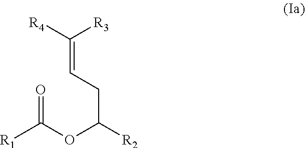
wherein
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NRSR₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me.

3. The use method according to claim 1, wherein the compound of formula (I) is the compound according to formula (Ib) 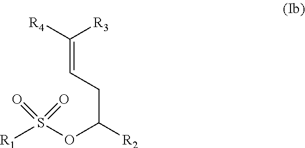
wherein
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NRsR₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me.

4. The method according to claim 1, wherein the compound according to formula (I) is selected from the group consisting of tetradeca-1,13-dien-4-yl acetate, tetradec-1-en-4-yl acetate, tridec-1-en-4-yl acetate, tetradec-1-en-4-yl benzoate, tridec-1-en-4-yl benzoate, tetradeca-1,13-dien-4-yl benzoate, tetradeca-1,13-dien-4-yl 2-naphthoate, tridec-1-en-4-yl 2-naphthoate, tetradec-1-en-4-yl 2-naphthoate, tetradec-1-en-4-yl 2-(naphthalen-1-yl)acetate, tridec-1-en-4-yl 2-(naphthalen-1-yl)acetate, tetradeca-1,13-dien-4-yl 2-(naphthalen-1-yl)acetate, tridec-1-en-4-yl 2-methylundecanoate, tetradec-1-en-4-yl 2-methylundecanoate, tetradec-1-en-4-yl dimethylcarbamate, methyl tetradec-1-en-4-yl carbonate, pentadec-3-en-6-yl acetate, pentadec-3-en-6-yl benzoate, tridec-1-en-4-yl 3,4,5-trimethoxybenzoate, tetradec-1-en-4-yl 3,4,5-trimethoxybenzoate, undec-1-en-4-yl benzoate, dodec-1-en-4-yl benzoate, tetradeca-1,13-dien-4-yl methanesulfonate and tetradeca-1,13-dien-4-yl 4-methylbenzenesulfonate.

5. A compound according to formula (I) 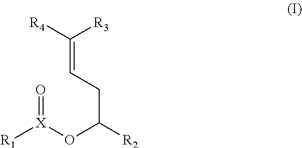
wherein
X is selected from C and S(═O);
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me,
with the provisothat the compound is not tetradec-1-en-4-yl acetate, tridec-1-en-4-yl acetate, tetradeca-1,13-dien-4-yl acetate, undec-1-en-4-yl benzoate, undec-1-en-4-yl pivalate, undec-1-en-4-yl acetate, methyl undec-1-en-4-yl carbonate, dodeca-1,11-dien-4-yl acetate, or dodec-1-en-4-yl acetate.

6. The compound according to claim 5, wherein the compound of formula (I) is the compound according to formula (Ia) 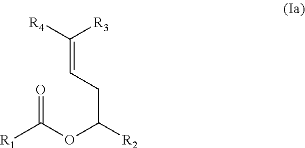
wherein
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me.

7. The compound according to claim 5, wherein the compound of formula (I) is the compound according to formula (Ib) 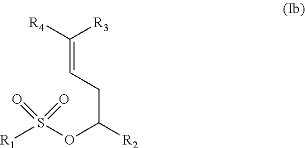
wherein
R₁ is selected from the group consisting of linear or branched C₁-C₁₁ alkyl; methoxy; ethoxy; phenyl with up to five substituents independently selected from methyl, ethyl, methoxy, ethoxy; naphthyl with up to four substituents independently selected from methyl, ethyl, methoxy, ethoxy; 1-naphthyl-methyl with up to two substituents independently selected from methyl, ethyl, methoxy, ethoxy; 1-(2-naphthyl)-eth-1-yl with up to two substituents independently selected from methyl, ethyl, methoxy, ethoxy; dialkylamines —NR₅R₆, with R₅ and R₆ being independently selected from the group consisting of C₁-C₄ alkyl;
R₂ is selected from the group consisting of linear C₇-C₁₀ alkyl and linear C₇-C₁₀ alkenyl with a terminal CC double bond;
R₃ is selected from the group consisting of H, Me, Et, phenyl, 2-naphthyl, 4-methoxyphenyl; and
R₄ is selected from the group consisting of H and Me

8. The compound according to claim 5, wherein the compound of formula (I) is selected from the group consisting of tetradec-1-en-4-yl benzoate, tridec-1-en-4-yl benzoate, tetradeca-1,13-dien-4-yl benzoate, tetradeca-1,13-dien-4-yl 2-naphthoate, tridec-1-en-4-yl 2-naphthoate, tetradec-1-en-4-yl 2-naphthoate, tetradec-1-en-4-yl 2-(naphthalen-1-yl)acetate, tridec-1-en-4-yl 2-(naphthalen-1-yl)acetate, tetradeca-1,13-dien-4-yl 2-(naphthalen-1-yl)acetate, tridec-1-en-4-yl 2-methylundecanoate, tetradec-1-en-4-yl 2-methylundecanoate, tetradec-1-en-4-yl dimethylcarbamate, methyl tetradec-1-en-4-yl carbonate, pentadec-3-en-6-yl acetate, pentadec-3-en-6-yl benzoate, tridec-1-en-4-yl 3,4,5-trimethoxybenzoate, tetradec-1-en-4-yl 3,4,5-trimethoxybenzoate, dodec-1-en-4-yl benzoate, tetradeca-1,13-dien-4-yl methanesulfonate and tetradeca-1,13-dien-4-yl 4-methylbenzenesulfonate.

9. A fragrance composition comprising a fragrance precursor which is at least one compound according to formula (I) as defined in claim 1.

10. A consumer product comprising a fragrance precursor which is at least one compound according to formula (I) as defined in claim 1 and a consumer product base.

11. The consumer product according to claim 10 wherein the consumer product is selected from home care and personal care products.

12. The consumer product according to claim 11 wherein the consumer product is selected from liquid detergent and shampoo.

13. A method of releasing a fragrance from a compound according to formula (I) as defined in claim 1, comprising exposing the compound to an environmental trigger.

14. A method of making a compound according to formula (I) as defined in claim 1, comprising the steps of:
a) reacting an aldehyde with allylmagnesium chloride; and
b) reacting the reaction product of step a) with acyl anhydride or chloride or sulfonyl chloride,
wherein steps a) and b) are performed as a one pot reaction.

15. A method to confer, enhance, improve or modify the hedonic properties of a fragrance composition or a consumer product, the method comprising adding to said composition or consumer product at least one compound according to formula (I) as defined in claim 1.

16. The method according to claim 15, comprising exposing said at least one compound to an environmental trigger.
